(19) United States
(12) Patent Application Publication
(10) Pub. No.: US 2017/0260500 A1
(43) Pub. Date: Sep. 14, 2017

(54) IMPROVEMENTS IN TISSUE PROCESSING

(71) Applicants: VIRGIN HEALTH BAK QSTP-LLC, Doha (QA); QATA FOUNDATON FOR EDUCATION,SCIENCE AND COMMUNITY DEVELOPMENT, Washington, DC (US)

(72) Inventors: Chris GOODMAN, Doha (QA); Kourosh SAEB-PARSY, Cambridge (GB)

(21) Appl. No.: 15/528,503

(22) PCT Filed: Nov. 20, 2015
(86) PCT No.: PCT/US2015/062011

§ 371 Date: May 20, 2017

(30) Foreign Application Priority Data
Nov. 21, 2014 (GB) 1420756.7

(51) Int. Cl. C12N 5/073; A01N 1/02

(52) U.S. Cl. CPC C12N 5/0605; A01N 1/0221; A01N 1/0278

(57) ABSTRACT

A method of preparing isolated body tissue (10) for cryogenic storage, the method comprising a step of actively infusing the body tissue (10) with a cryoprotectant (20). Additionally, a kit for preparing umbilical cord tissue (10) for cryogenic storage, comprising a cryoprotectant (20) and infusion means (22) for actively infusing the umbilical cord tissue (10) with the cryoprotectant (20).

### TECHNICAL FIELD

The invention relates to a method of preparing body tissue for cryogenic storage, particularly umbilical cord tissue.

### BACKGROUND ART

Umbilical cord blood is well-known to contain haematopoietic stem cells (“HSCs”), and it is known to harvest cord blood from an umbilical cord shortly after birth and to store the cord blood (and HSCs therein)—typically cryogenically—for therapeutic use at a later stage. In recent years, umbilical cord tissue has also been identified as a rich source of stem cells. Mesenchymal stem cells (“MSCs”) are present in the cord tissue-in and on the umbilical cord vein, the umbilical cord arteries and in the Wharton's jelly, notably in the perivascular Wharton's jelly. MSCs have emerged as major candidates in the field of cell-based therapies, particularly in regenerative medicine, and it is desirable to store MSCs for later use by the donor, family member or other allogeneic recipient. Accordingly, improved devices and methods for processing and storing umbilical cords, to enable access to viable stem cells when required, have become highly desirable.

The therapeutic potential of umbilical cord-derived MSCs is discussed in Dalous et al, Pediatric Research (2012) 71, 482-490. Typically, MSCs are obtained from the umbilical cord by dissecting the cord into small pieces and enzymatically digesting these pieces, typically with collagenase alone or in combination with trypsin and hyaluronidase. The isolated stem cells are then stored for later use.

WO-A-2011/073388 describes a method of separating the umbilical vascular tissue from the Wharton's jelly, followed by the separation of dissociated (stem) cells from the jelly matrix and the independent separation of stem cells from the vascular tissue, and cryopreservation of each population of separated dissociated cells.

There is currently no standardised procedure for extracting stem cells from umbilical cord tissue. Therefore, it may be advantageous to store whole umbilical cord tissue so that the stem cells may be extracted at a later stage. The current approach to storing whole tissue in this way is to bathe, soak or immerse the tissue in a dimethyl sulfoxide (DMSO) solution (5-30% v/v, typically 10%v/v) containing human serum albumin (HSA) or Fetal Bovine Serum (FBS) at 4° C. for approximately 40 to 90 minutes, and then preserve the tissue cryogenically. An example of this technique is described in WO-A-2007/071048. However, cryoprotectants such as DMSO are toxic to the cells, and so the current methods of preparing the tissue for storage are not ideal.

An improved method is therefore needed for preparing body tissue for storage, in particular tissue comprising stem cells such as umbilical cord tissue.

### DISCLOSURE OF INVENTION

It has surprisingly been found by the inventors that actively infusing body tissue with a cryoprotectant reduces the time taken to prepare the body tissue for cryogenic storage. This is of particular use in preparing umbilical cord tissue for cryogenic storage.

The active infusion of the tissue with the cryoprotectant reduces damage to and degeneration of the tissue that can occur as a result of prolonged incubation with the cryoprotectant, thereby improving the quality of the preserved tissue. The body tissue typically comprises stem cells, and the method can increase the yield of viable stem cells that can be isolated from the cryogenically stored tissue. The method of the invention thereby aids the post-thaw viability of cryogenically-frozen tissue and cells obtainable from that tissue.

In accordance with a first aspect of the invention, there is provided a method of preparing isolated body tissue for cryogenic storage, the method comprising a step of actively infusing the body tissue with a cryoprotectant.

The cryoprotectant may be actively infused directly to an internal region of the body tissue. An internal region may be the lumen, intravascular space or interstitial space. For example, cryoprotectant can be pumped into the lumen or intravascular space, or injected into the interstitial space.

The body tissue may be actively infused with the cryoprotectant using an infusion means, such as a pressure-exerting means. The pressure-exerting means can be any means which is able to exert pressure, or a force, on the cryoprotectant to infuse actively the body tissue with cryoprotectant. The pressure-exerting means is able to force the cryoprotectant into the body tissue. The infusion means may comprise at least one of a pump (e.g. a perfusion pump), a syringe, a vacuum, a coil spring, a leaf spring, a Belville spring or a resilient element. The infusion means may be activated manually, automatically, or electronically.

Cryoprotectants are well-known. In one embodiment, the cryoprotectant comprises DMSO. Alternatively or additionally, the cryoprotectant may comprise glycerol.

In one embodiment, the body tissue comprises stem cells, typically Mesenchymal Stem Cells (MSCs). The tissue comprising stem cells may, in a further embodiment, comprise at least a portion of an umbilical cord, for example at least a portion of an umbilical cord vein, an umbilical cord artery or Wharton's jelly, typically perivascular Wharton's jelly.

Typically, the body tissue is an intact piece of umbilical cord, i.e. umbilical cord that has not been divided into its constituent parts and which comprises the umbilical cord vein, two umbilical cord arteries and Wharton's jelly. When the tissue is umbilical cord tissue, the cryoprotectant may typically be actively infused directly into the lumen of the umbilical cord vein and/or the lumen of one or both umbilical cord arteries. Alternatively or additionally, the cryoprotectant may be actively infused through the amniotic epithelium that forms the outer layer of the umbilical cord, so that the cryoprotectant is infused directly into the umbilical cord tissue (i.e. directly into the Wharton's jelly and/or vasculature).

The method may further comprise soaking, bathing or submerging (partially or completely) the actively infused body tissue in a further quantity of a cryoprotectant, which may be the same cryoprotectant, or a different cryoprotectant, that is actively infused into the tissue. This step can occur before, during or after the active infusion step. Typically, it is performed contemporaneously with, or consecutively after, the active infusion step. For example, the body tissue may be injected or pumped with cryoprotectant immediately prior to being placed in a container of cryoprotectant, or alternatively the body tissue may be placed in a container of cryoprotectant and then injected or pumped with cryoprotectant. The container is typically suitable for cryogenic storage, such as a sealable sterile polypropylene container.

The method may comprise incubating the body tissue in the cryoprotectant for less than approximately 40 minutes or less than approximately 30 minutes. Typically, the incubation period is 20 minutes or less, for example between approximately 10 minutes and approximately 20 minutes. The incubation is carried out at a temperature less than 37° C., i.e. less than human body temperature. Typically, the optional incubation step occurs at between 1° C. and 10° C., more typically between 2° C. and 5° C., for example at approximately 4° C.

Once the body tissue has been treated according to the invention, it can be cryogenically frozen and stored. In the embodiment where the incubation step is carried out in a container suitable for cryogenic storage, then the container comprising the body tissue and cryoprotectant can be closed or sealed if necessary, and cryogenically frozen, e.g. by placing into liquid nitrogen.

In accordance with a second aspect of the invention, there is provided body tissue that has been prepared by the method of the first aspect of the invention.

A third aspect of the invention provides a stem cell which has been isolated from the body tissue of the second aspect. In one embodiment, the stem cell is a mesenchymal stem cell.

In accordance with a fourth aspect of the invention, there is provided a method of storing umbilical cord tissue, comprising:
(i) actively infusing umbilical cord tissue with a cryoprotectant; and
(ii) cryogenically storing the cryoprotectant-infused umbilical cord tissue for at least one year.

In one embodiment of the fourth aspect, the method further comprises the steps of:
(iii) thawing all or part of the stored umbilical cord tissue; and
(iv) isolating stem cells from the thawed umbilical cord tissue.

In accordance with a fifth aspect of the invention, there is provided a kit for preparing umbilical cord tissue for cryogenic storage, comprising:
(i) a cryoprotectant; and
(ii) infusion means for actively infusing the umbilical cord tissue with the cryoprotectant.

The infusion means may comprise a pressure-exerting means. The kit may also comprise a device for collecting or harvesting blood comprising a housing configured to receive a blood source and a first collection means in communication with the housing, wherein the housing comprises a first output configured to allow passage of blood extracted from the blood source and/or at least a portion of the blood source therethrough.

### BRIEF DESCRIPTION OF DRAWINGS

The present invention will now be described with reference to the accompanying drawings, in which:

FIG. 1 shows a cross-sectional view of a typical umbilical cord for use in the present invention;

FIG. 2 shows a perspective view of the umbilical cord shown in FIG. 1; and

FIGS. 3A, 3B and 3C show a portion of an umbilical cord being processed according to embodiments of the present invention.

FIGS. 4 and 5 show a device for collecting cord blood that can be used to prepare umbilical cord tissue before use in the present invention.

### BEST MODES FOR CARRYING OUT THE INVENTION

The present invention relates to methods for preparing biological tissue, particularly umbilical cord tissue, for cryogenic storage.

The invention involves the active infusion of a cryoprotectant into body tissue. Active infusion applies a force to the cryoprotectant, to infuse the cryoprotectant actively into the tissue and provide an increased rate of uptake into the tissue compared to the uptake that would occur in the absence of that force. This is in contrast to the passive application of cryoprotectant that is known in the art, which involves soaking, bathing or submerging the tissue in cryoprotectant and which relies on the cryoprotectant simply soaking into the tissue.

Active infusion can, for example, refer to the use of a pressure-exerting means to infuse the tissue with a cryoprotectant. Typically, the method of the invention injects or pumps a cryoprotectant directly into the tissue. This active infusion provides an improvement over the passive diffusion that is provided in the prior art methods of soaking, immersing or submersing the tissue in the cryoprotectant. Cryoprotectants are typically toxic and one improvement is that the time required for the tissue to be in contact with the toxic cryoprotectant is reduced when the method of the invention is used. The active infusion of the tissue with a cryoprotectant thereby reduces damage to and degeneration of the tissue as a result of incubation with the cryoprotectant, thereby improving the quality of the preserved tissue.

When the cryoprotectant is injected, this may be achieved using a syringe; the syringe may comprise a needle (e.g. a hypodermic needle) or may be a needle-less syringe, typically powered by compressed air or gas.

Isolated Body Tissue

Any body tissue can be prepared for storage according to the invention. The body tissue is isolated, i.e. no longer part of the human or animal body. The tissue is isolated from the body before the method of the invention takes place. The tissue is typically human body tissue. The tissue is typically soft tissue. The body tissue typically contains adult stem cells. The stem cells are typically mesenchymal stem cells. Alternatively, the stem cells may be haematopoietic stem cells or neural stem cells.

The tissue is typically processed within about 24 hours from the time is it removed from the body, more typically within 12 hours. In one embodiment, the tissue is processed and stored without delay, for example within one hour of being removed from the body, or within 30 minutes of being removed from the body.

Suitable body tissues include the umbilical cord, placenta and the amniotic membrane.

The cryoprotectant may be actively infused directly to an internal region of the body tissue. An internal region is not exposed to the external environment when the tissue is intact. An internal region may be the lumen, intravascular space or interstitial space of a given tissue.

Cryoprotectant may be pumped into the lumen or intravascular space; for example, the cryoprotectant may be pumped or injected into the lumen of a blood vessel (i.e. into the vascular space) or into the lumen of a lymph vessel. Cryoprotectant may be injected into the interstitial space; active infusion into the interstitial space will typically be achieved by injection across the outer membrane of the tissue, if present.

When the cryoprotectant is delivered to an open-ended body tissue, for example to the lumen of a vein or artery, one end of the tissue may be closed or sealed during the active infusion step. This prevents the infused cryoprotectant from flowing out of the tissue and allows cryoprotectant that is actively infused into lumen, typically by infusion into the other (open) end of the lumen, to accumulate in the lumen and in the surrounding tissue. Typically, the cryoprotectant accumulates in the lumen and exerts a pressure on the lumen walls, so that the cryoprotectant is forced across the lumen wall and into the interstitial space of the tissue. The tissue may be sealed temporarily using a clamp or clip, or may be sealed permanently, for example by cauterisation.

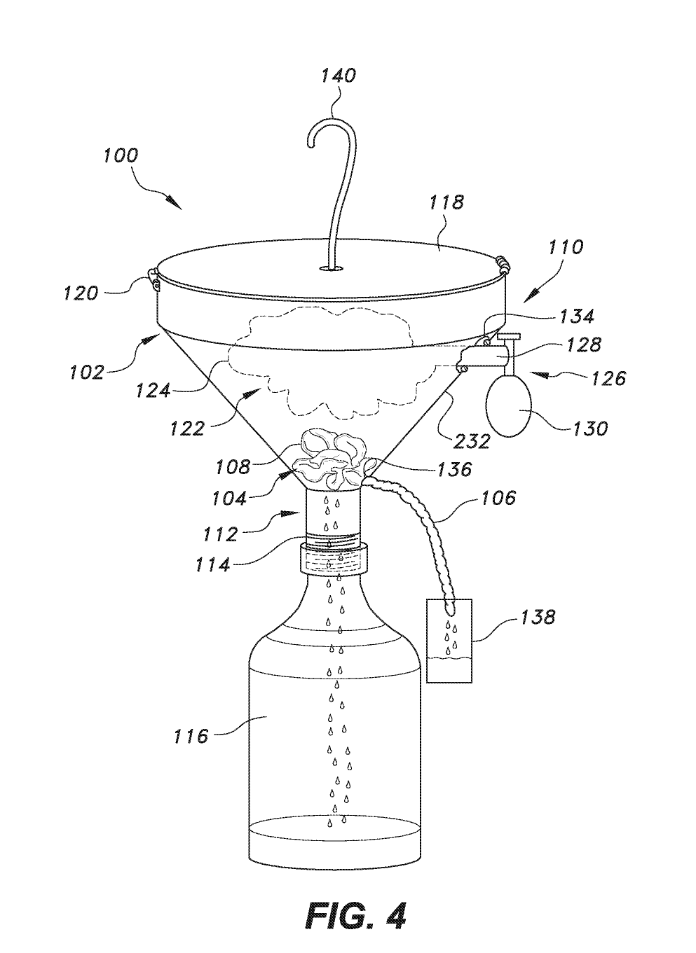

This approach may advantageously be applied to umbilical cord tissue, where one end of the cord (or portion or segment thereof) can be clamped or clipped while the cryoprotectant is actively infused, typically by injection or pump, into the open (unclipped) end of the umbilical vein and/or artery. Optionally, the cryoprotectant may be actively infused into the umbilical vein and one or both arteries, until the vein and artery/arteries are full of cryoprotectant, and then the end of the vein/artery that was the site of infusion is also closed or sealed (e.g. with a clamp or clip), thereby retaining the cryoprotectant under pressure in the vein and artery/arteries. The pressure of the cryoprotectant against the vasculature walls (akin to turgor pressure) forces the cryoprotectant through the vascular epithelium and into the umbilical cord tissue. Optionally, the cord that comprises cryoprotectant and is closed at both ends may be submerged in a further amount of cryoprotectant, as shown in FIG. 3C.

Cryogenic Storage

Cryogenic storage of tissues is well-known in the art and involves storing the tissue at very low temperatures, typically using a cryogenic substance such as liquid nitrogen, liquid air, liquid natural gas, liquid carbon dioxide or any other cryogen. Cryogenic storage is typically t a temperature lower than −150° C., more typically lower than −196° C. (which is the boiling point of liquid nitrogen).

Cryogenic storage is well known in the art. Typically, the body tissue and cryoprotectant are placed in a container made of a non-brittle sterilisable polymer such as polypropylene. This container is then frozen in a cryogen such as liquid nitrogen, where it is retained until needed. In one embodiment of the present invention, the container in which the body tissue is incubated (by bathing or submerging in cryoprotectant) for less than 40 minutes and typically 10 to 20 minutes, is the container in which the tissue is cryogenically frozen.

The method of the invention prepares tissue for cryogenic storage, typically long-term cryogenic storage. The tissue typically contains stem cells, which it is useful to store long-term so that viable stem cells can be accessed when needed, for example when needed for regenerative medicine, to treat the donor, family member or other allogeneic person.

Once prepared according to the method of the invention, the tissue is typically cryogenically stored long-term. This long-term storage is typically for at least one year, more typically at least five years and yet more typically at least ten years. Storage for at least 15 years or at least 20 years, for example at least 25 years or at least 50 years is also within the scope of the phrase “long-term”.

Methods of thawing cryogenically stored tissue to obtain viable cells, at the time when the tissue and cells are needed, are well-known in the art. Briefly, the tissue is typically thawed in a water bath at between 20 to 40° C., for example 37° C. Once thawed, the tissue is typically transferred to a different container and washed to dilute or remove the cryoprotectant. The washing may be performed using cool (e.g., refrigerated, such as 4° C.) liquid, such as water or buffered saline, e.g., PBS, by immersing the tissue in the cool liquid. Vigorous washing of the tissue is often avoided, so that shock or damage to the cells is minimized The immersed tissue can be retained in a refrigerator for another period to permit further dilution and replacement of the cryoprotectant by water, and then still further diluted by addition of further cooled liquid.

The resulting restored tissue can then be used to recover viable cells, such as stem cells, resident within the tissue, optionally using the known techniques for recovering viable cells from fresh cord tissue.

Cryoprotectants

The tissue is actively infused with a cryoprotectant. A cryoprotectant is a substance that is provided to cells before freezing and yields a higher post-thaw survival of viable cells than can be obtained in its absence. Cryoprotectants are well-known in the art and typically protect biological tissue from freezing damage caused by ice formation. Various cryoprotectants may be used according to the invention. Typical cryoprotectants are described below.

Some cryoprotectants permeate the cell membrane and protect the cell from damage during freezing. These permeating cryoprotectants include: glycols such as ethylene glycol, propylene glycol and glycerol; butanediol e.g. 2,3-butanediol; and Dimethyl sulfoxide (DMSO; [CH₃]₂SO). DMSO is a conventional cryoprotectant that is often used to bathe umbilical cord tissue prior to storage in liquid nitrogen. Cryoprotectants typically comprise 5% to 30% (v/v) DMSO, for example 10% (v/v) or 20% DMSO. In one embodiment, the cryoprotectant comprises 10% (v/v) DMSO.

Cryoprotectants are also known that do not permeate the cell membrane, including: Dextran, e.g. Dextran 40; disaccharides such as sucrose or trehalose; percoll; polyethylene glycol (PEG); and polyvinypyrrolidone (PVP).

Combinations of cryoprotectants may be used. For example, the concentration of DMSO required can be reduced if be combined with disaccharides such as trehalose or sucrose (Rodrigues et al, Cryobiology 56(2) April 2008: p 144-151). The disaccharide is typically present at 0.1M with 10% DMSO. A mixture of DMSO and Dextran is also known as an effective cryoprotectant, as is a mixture of DMSO and glycerol, and these mixtures can be used according to the invention.

The cryoprotectant may comprise additional components such as plasma, serum or a serum component such as fetal bovine serum (FBS), Bovine Serum Albumin (BSA), or Human Serum Albumin (HSA). The serum or plasma is typically human and may be obtained from the mother. In one embodiment, the cryoprotectant comprises autologous serum (from the mother) containing 10%(v/v) DMSO.

The active infusion of the tissue with a cryoprotectant is performed at a temperature less than 37° C., i.e. less than body temperature. Typically, the infusion step occurs at between 1° C. and 10° C., more typically between 2° C. and 5° C., for example at approximately 4° C.

Preparation of Umbilical Cord Tissue for Cryogenic Storage

An exemplary tissue that can be prepared according to the invention is umbilical cord tissue. The umbilical cord tissue that is used according to the invention, is postpartum tissue that has been removed (typically cut) from the infant.

The umbilical cord tissue may comprise at least a portion of an umbilical cord, for example at least a portion of an umbilical cord vein, a portion of an umbilical cord artery or Wharton's jelly. Long-term storage of the umbilical cord is desirable because mesenchymal stem cells have been identified throughout the umbilical cord, including in the sub-endothelial layer of the umbilical vein and arteries, and in the Wharton's jelly (in particular the perivascular Wharton's jelly). Umbilical cord MSCs are obtained from term umbilical cord after parturition, and are not embryonic stem cells. Umbilical cord MSCs are not capable of developing into an embryo. Obtaining and using umbilical cord blood MSCs does not involve the destruction of human embryos, and obtaining umbilical cord MSCs avoids the controversy and ethical considerations surrounding the provision and use of human embryonic stem (ES) cells.

Typically, the body tissue is an intact piece of umbilical cord, i.e. that has not been divided into its constituent parts, by mechanical or enzymatic means. Intact umbilical cord tissue comprises the umbilical cord vein, two umbilical cord arteries and the Wharton's jelly, surrounded by amniotic epithelium. Intact umbilical cord tissue has not been dissected, cut into pieces or minced.

The storage of intact umbilical cord tissue allows for all of the potentially useful cells, in particular stem cells, to be cryogenically stored without losing yield by extraction prior to freezing. Methods for isolating stem cells from tissues are currently subject to significant variation in yield and so it is beneficial to store the umbilical cord as whole tissue. This cryogenically-stored tissue, or a portion of the cryogenically-stored tissue, can be accessed when needed and the stem cells isolated at that point in time, which may improve the yield and/or functionality of the cells that are obtained.

The cryoprotectant may typically be actively infused directly into the lumen of the umbilical cord vein and/or the lumen of one or both umbilical cord arteries. Infusion directly into the lumen may be achieved by pumping or injecting cryoprotectant into an open end of the vein to or artery, or by accessing the lumen of the artery by injecting or pumping through the umbilical cord tissue. Alternatively or additionally, the cryoprotectant may be actively infused across the amniotic epithelium that forms the outer layer of the umbilical cord, so that the cryoprotectant is infused directly into the umbilical cord tissue (i.e. directly into the Wharton's jelly and/or vasculature). When the cryoprotectant is injected into the umbilical cord, this may be achieved using a syringe; the syringe may comprise a needle (e.g. a hypodermic needle) or may be a needle-less syringe, typically powered by compressed air or gas.

In one embodiment, an intact umbilical cord is placed into a cryoprotectant bath comprising 10% (v/v) DMSO, at 4° C., and cryoprotectant comprising 10% (v/v) DMSO is injected into the umbilical cord vein or artery lumen using a syringe. The cryoprotectant may optionally comprise autologous serum or plasma (from the mother). The cord is then incubated in the cryoprotectant for ten to twenty minutes at 4° C., before being cryogenically frozen.

Preparation of Umbilical Cord Tissue

When the body tissue is umbilical cord tissue, some or all of the umbilical cord blood may optionally be removed from the cord before performing the method of the invention. For example, the cord blood may first be harvested for (separate) storage, and the cord which is substantially free of cord blood (but may of course comprise a residual amount of cord blood cells) then prepared for cryopreservation according to the invention. In this way, the cord blood and cord tissue can be harvested and stored.

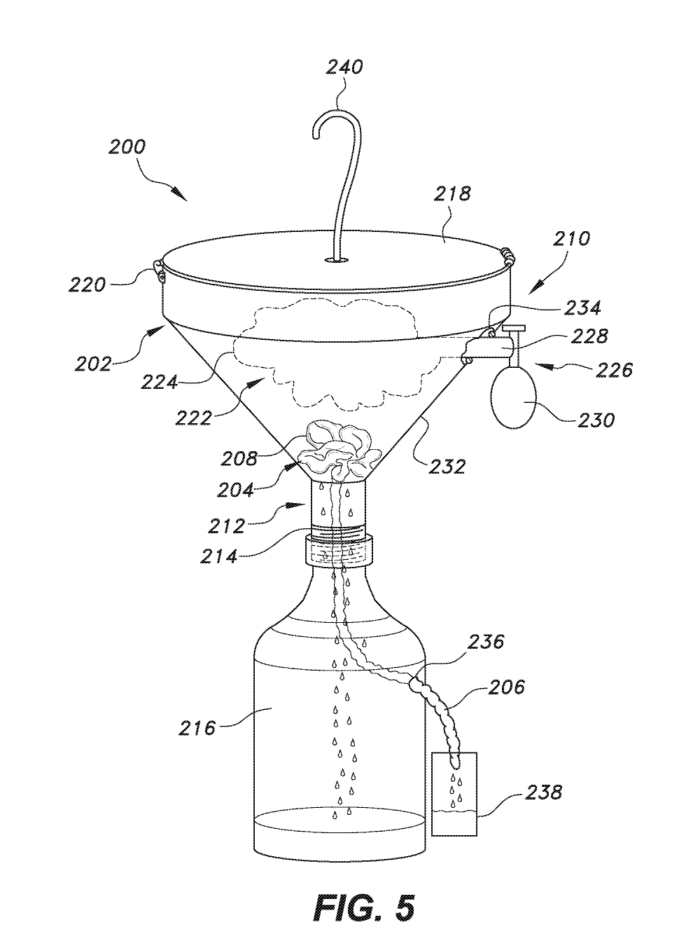

The collection of umbilical cord blood is known in the art and a typical device that can be used to collect the cord blood is described in WO-A-2014/057353 (Virgin Health Bank QSTP-LLC, incorporated herein by reference). Exemplary devices of this kind are shown in FIGS. 4 and 5, below. This device comprises a housing configured to receive a blood source and a first collection means in communication with the housing, wherein the housing comprises a first output configured to allow passage of blood extracted from the blood source and/or at least a portion of the blood source therethrough. The device may be configured to allow for collection of blood from the blood source using a gravitational force. The first output of the device may be configured to allow passage of waste from the blood source therethrough, and the device may further comprise a second output configured to allow passage of at least a portion of the umbilical cord therethrough. By providing separate outputs for the waste and the blood source, waste can be easily separated from useful products. For example, when the blood source is an umbilical cord and placenta, a portion of the cord can be fed through the second output and umbilical cord blood can be collected. Waste from the placenta and cord can be passed through the first output and collected for disposal. A first collection means may be in communication with the housing via the first output. The first collection means may threadingly or pushingly engage the housing at the first output. The device may further comprise a second collection means in communication with the second output; the housing may comprise the second output. The device may further comprise a pressure-exerting means configured in use to exert pressure on the blood source. The provision of a pressure exerting means allows pressure to be exerted on the blood source to force blood out of the blood source effectively and efficiently. At least a portion of the pressure-exerting means may be located within the housing. The pressure-exerting means may comprise an inflatable located within the housing and an inflation means in communication with the inflatable which may extend outside the housing, wherein the inflation means may comprise a conduit configured to transfer fluid, for example, from outside the housing into the inflatable to inflate the inflatable thereby exerting pressure on the blood source.

Once the blood has been collected using a cord blood collection device, the cord tissue can be actively infused with a cryoprotectant according to the present invention. Optionally, the umbilical cord is removed from the cord blood collection device before it is actively infused with cryoprotectant.

In one embodiment, the umbilical cord remains in the cord blood collection device while the active infusion step is carried out. In this way, the umbilical cord can be placed in the device and the cord blood harvested, and then the umbilical cord can be actively infused while connected to the device. This provides a simple method for harvesting both umbilical cord tissue and umbilical cord blood.

For example, in FIGS. 4 and 5, the umbilical cord 106, 206 (whether attached to the blood collection device or not) can be injected or pumped with a cryoprotectant. In one embodiment, the cord is drained of the cord blood and then the lower part of the cord 106, 206 is closed (e.g. with a clip or a clamp, as discussed above). Cryoprotectant can then be injected or pumped into the cord according to the invention, into one or both arteries, the vein and/or the interstitial space. Once the cryoprotectant has been actively infused, a second clip or clamp may be applied to the upper part of the umbilical cord, to provide a sealed cord that contains cryoprotectant. This sealed cord can then optionally be placed in a further amount of cryoprotectant and incubated, typically at between 1° C. and 10° C. for 30 minutes or less, typically 20 minutes or less.

An embodiment of the invention, wherein umbilical cord tissue is prepared for cryogenic storage, is now described with reference to the figures.

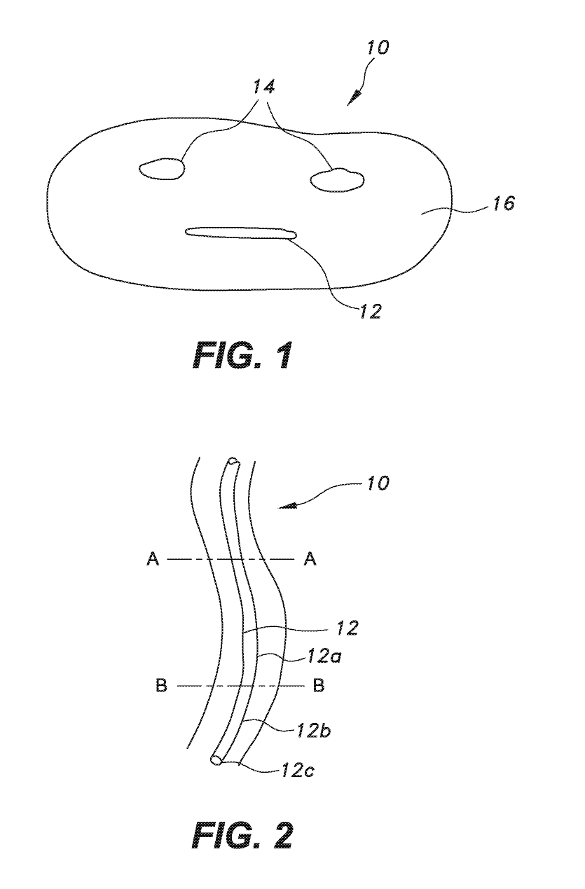

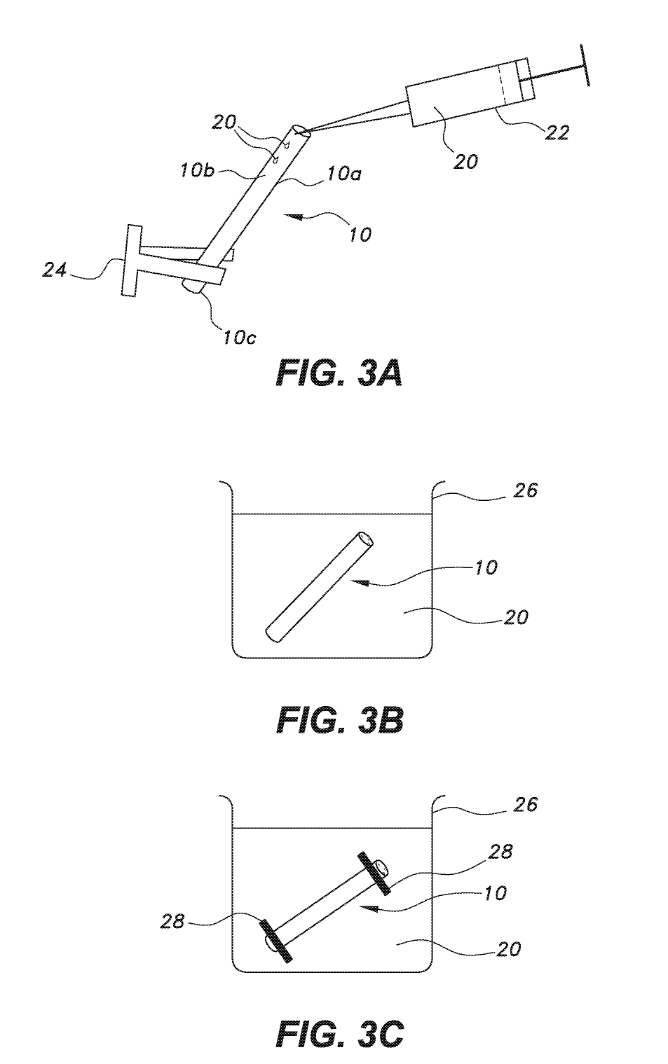

FIG. 1 shows a cross-sectional view of a typical umbilical cord 10. The cord 10 comprises an umbilical cord vein 12 which carries oxygenated blood to a foetus, two umbilical arteries 14 which carry deoxygenated blood from the foetus, and Wharton's jelly 16 which is a gelatinous substance that protects and supports the vein 12 and arteries 14. FIG. 2 shows a perspective view of the same umbilical cord 10 which has been cut-away to show the location of the umbilical cord vein 12.

The entire umbilical cord may be prepared for storage. Alternatively, a section of the intact cord tissue may be prepared for storage. A section may be obtained by make a sectional cut along the lines A-A and B-B shown in FIG. 2, and then removing the desired section of umbilical cord tissue from the umbilical cord 10. This section comprises intact umbilical cord tissue: the vein, both arteries, Wharton's jelly, all surrounded by the epithelium.

An exemplary method for preparing the intact umbilical cord tissue 10 for long-term cryogenic storage will now be described with reference to FIGS. 3A and 3B. However, the skilled person will understand that the same method could be applied to the an isolated part of the umbilical cord, i.e. the umbilical cord vein 12, umbilical cord arteries 14 and/or Wharton's jelly 16, or indeed any body tissue containing stem cells.

FIGS. 3A and 3B show an exemplary method for preparing a portion of intact umbilical cord tissue 10 for long-term cryogenic storage.

As shown in FIG. 3A, intact umbilical cord tissue 10 is actively infused with a cryoprotectant 20, such as DMSO (10%v/v) at 1° C.-10° C., typically 2° C.-5° C., and more typically 4° C. The skilled person will understand that any suitable cryoprotectant could equally be used. In the exemplary embodiment shown in FIG. 3A, the cryoprotectant 20 is introduced into the umbilical vein lumen 10b by active infusion. To assist with this infusion, one end 10c of the cord 10 is held closed using a suitable clamp 24. Active infusion can, for example, refer to the use of a pressure-exerting means to infuse the tissue with cryoprotectant 20. In other words, a pressure or force is exerted on the cryoprotectant 20 to force the cryoprotectant 20 into the tissue 10. In the exemplary embodiment shown in FIG. 3A, the active infusion is achieved using a syringe 22. However, any alternative active infusion means, or pressure-exerting means, could equally be used, such as a pump (e.g. a perfusion pump), a vacuum, a coil spring, a leaf spring, a Belville spring or a resilient element. The syringe 22 shown in FIG. 3A is intended for manual activation (e.g. by applying pressure to a plunger), but the infusion means could also be activated automatically and/or electronically.

The step shown in FIG. 3A can be applied to any other body tissue having an interior lumen, cavity or space. As well as applying the cryoprotectant 20 to any interior lumens, cavities or other spaces within the body tissue, the infusion means 22 can be used to force cryoprotectant 20 into the bulk tissue itself, i.e. into the interstitial space. This is particularly appropriate in the case of body tissue which does not comprise any interior lumens, cavities or other spaces, and active infusion is still effective when applied to such tissue.

As shown in FIG. 3B, once the cord tissue 10 or other tissue has been actively infused with cryoprotectant 20 as described above with reference to FIG. 3A, the entire cord tissue 10 is placed in a suitable receptacle 26 (e.g. an incubator, typically a polypropylene container that is suitable for cryogenic storage) and partially or wholly submerged and soaked in another quantity of cryoprotectant 20, which can be the same cryoprotectant used for active infusion (shown in FIG. 3A) or a different cryoprotectant. The submerged cord tissue 10 is then incubated. In an exemplary embodiment, the cryoprotectant used for incubation is DMSO, for example 5% DMSO or 10% DMSO, in which case the vein 12 is incubated at 1° C.-10° C., typically 2° C.-5° C., and more typically 4° C. However, the skilled person will understand that other cryoprotectants, such as Dextran40 or glycerol, could equally be used. A function of the cryoprotectant is to prevent the formation of ice crystals when the whole tissue is stored cryogenically (i.e. when it is frozen), as ice crystals can damage the tissue and reduce the yield of MSCs which can be obtained from the tissue after the tissue has been thawed.

FIG. 3C shows the incubation in cryoprotectant 20 of an umbilical cord 10 that has been actively infused with cryoprotectant 20 (as shown in FIG. 3A) and has both ends closed using a clip 28. This cord is therefore turgid with cryoprotectant that has been actively infused, and is bathed in a further quantity of cryoprotectant.

The skilled person will understand that preferred incubation times may vary, but the incubation time will typically be less than approximately 40 minutes, preferably less than approximately 30 minutes, more preferably less than approximately 20 minutes, and more preferably between approximately 10 minutes and approximately 20 minutes.

Once the tissue has been incubated, it is ready for cryogenic storage. Incubation followed by cryogenic storage permits umbilical cord tissue, or other body tissue, to be preserved as whole tissue until it is needed, for example when stem cells present in the tissue need to be extracted. Since the available methods for extracting stem cells from whole tissue are continually being improved, effective storage means that the best possible method for extracting the stem cells can be employed at the time that the MSCs are needed. The particular preparation period described above, which includes actively infusing a portion of body tissue with cryoprotectant, as opposed to simply submerging this tissue in cryoprotectant, means that interior portions, concealed portions, or difficult to access portions of the body tissue can be infused with cryoprotectant effectively and efficiently resulting in optimal post-thaw stem cell extraction. Moreover, the active infusion of portions of the body tissue, such as an umbilical vein, which are concealed, hidden or difficult to access (e.g. the interior of the umbilical cord vein) means that incubation times can be reduced significantly when compared with simply submerging or “soaking” the whole tissue umbilical cord vein, or other body tissue, in a cryoprotectant.

Device for Collecting Umbilical Cord Blood Prior to Active Infusion

As explained above, some or all of the umbilical cord blood may optionally be removed from the cord before performing the active infusion of cryoprotectant according to the present invention. FIGS. 4 and 5 show devices that may be used to harvest the cord blood.

FIGS. 4 and 5 show configurations of a blood-collecting device 100, 200. The device comprises a housing 102, 202 configured to receive a blood source 104, 204. In the figures, the blood source 104, 204 is an umbilical cord 106, 206 and placenta 108, 208 which have previously been detached from a human body.

The housing 102, 202 shown in the figures has a substantially conical portion 108, 208 and generally takes the form of a funnel i.e. having a wide portion at a top end 110, 210 of the housing 102, 202 and tapering gradually inwards to a narrow bottom end 112, 212 of the housing 102, 202. The skilled person will understand that a housing having any other suitable shape (e.g. frustoconical, bowl-shaped, cylindrical) could equally be used. The housing 102, 202 comprises a first output 114, 214, which takes the form of a gap or hole located at the narrow end of the housing 102, 202. Alternatively, the first output 114, 214 can be a removable cover, a permeable membrane, a thinning, or any other output through which a blood source and/or waste from a blood source could pass. In some embodiments, the housing is movable between a deployed configuration and a collapsed configuration, for example, in a concertinaing manner.

A first collection means 116, 216 is in communication with the housing 102, 202 via the first output 114, 214. FIGS. 4 and 5 show the first collection means 116, 216 in threaded engagement with (i.e. screwed onto) the housing 102, 202 at the first output 114, 214. The first collection means 116, 216 can alternatively pushingly engage the housing 102, 202 at the first output 114, 214. A closure 118, 218, such as a lid and/or membrane, is also provided to close or seal the housing 102, 202. In some embodiments, the closure 118, 218 is hingedly attached to the housing 102, 202 and/or releasably attached to the housing 102, 202 by means of a latch or clip 120, 220. The first collections means 116, 216 can, for example, take the form of a wind sock, a flask, or any other suitable bag or container.

The device also comprises a pressure-exerting means 122, 212, at least a portion of which is contained within the housing 102, 202. The pressure-exerting means 122, 212 shown in FIGS. 4 and 5 comprises an inflatable, such as an inflatable sack 124, 224, located within the housing 102, 202 and an inflation means 126, 226 in communication with the inflatable 124, 224 and extending outside the housing 102, 202. The inflatable can alternatively be an inflatable balloon, inflatable pouch, or any other inflatable. The inflation means 126, 226 comprises a conduit 128, 228 configured to transport fluid from outside the housing 102, 202 into the inflatable 124, 224 to inflate the inflatable. The inflation means also optionally comprises a pump 130, 230 located outside the housing 102, 202 and in communication with the conduit 128, 228, wherein the pump 130, 230 is configured to force fluid through the conduit 128, 228 and into the inflatable 124, 224. The figures show the conduit 128, 228 passing through a wall 132, 232 of the housing 102, 202. However, the skilled person will understand that the conduit can also pass through the housing closure 118, 218 or any other portion of the housing 102, 202. It is desirable to ensure a tight seal between the conduit 128, 228 and the closure or housing. A grip or stopper 134, 234, for example a rubber grip or stopper, surrounding the conduit 128, 228 and engaging with the closure or housing can be provided to ensure that an airtight and sterile environment is maintained within the housing 102, 202.

The fluid used to inflate the inflatable 124, 224 may be a gas, such as air, in which case the inflatable can be an airbag and the pump can be an air pump. Alternatively, a liquid, such as water, can be used to inflate the inflatable 124, 224.

As described above, the housing 102, 202 comprises a first output 114, 214. In the exemplary embodiments shown in the figures, the first output is configured to allow passage of waste from the blood source 104, 204 therethrough. The device 100, 200 also comprises a second output 136, 236, which is configured to allow passage of at least a portion of the blood source 104, 204 therethrough. For example, if the blood source is an umbilical cord 106, 206 and/or placenta 108, 208, the second output 136, 236 is configured to allow passage of the umbilical cord 106, 206 therethrough, meaning that umbilical cord blood passes along or through the umbilical cord, through the second output 136, 236. A second collection means 138, 238 is provided to collect the blood extracted from the blood source. The second collection means 138, 238 is typically a bag or flask, although any other suitable collection means can equally be used.

In the configuration shown in FIG. 4, the housing 100 comprises the second output 136.

In the configuration shown in FIG. 5, the first collection means 216 comprises the second output 236. In other words, the umbilical cord 206 is passed through the first output 214 into the first collection means 216, and is then passed through the second output 236 and into the second collection means 238.

In both configurations shown in the figures, the second output is smooth and rounded without any sharp edges so as not to risk tearing the umbilical cord and spilling umbilical cord blood. The second output is a gap or hole on a wall of the housing through which an umbilical cord can pass. Alternatively, the second output is a thinning or a removable cover.

An exemplary method of using the device 100, 200 to harvest cord blood will be described with reference to the figures. The closure 118, 218 is moved to an open position or removed from the housing 102, 202, and a blood source 104, 204, such as an umbilical cord 106, 206 and/or a placenta 108, 208 is placed into the housing 102, 202. The umbilical cord 106, 206 is passed through the second output 136, 236 so that an end of the umbilical cord extends outside the housing. In some embodiments (not shown), a cord clamp is attached to the umbilical cord, in which case the first and/or second output is sufficiently large to allow passage of a cord clamp therethrough. If the device shown in FIG. 4 is used, the umbilical cord 106 is passed directly through the second output 136 into the second collection means 138. If the device shown in FIG. 5 is used, the umbilical cord 206 is passed through the first output 214 into the first collection means 216, and is subsequently passed through the second output 236 into the second collection means 238. In both configurations, the placenta 108, 208 remains within the housing 102, 202 with the inflatable 124, 224 of the pressure-exerting means. The skilled person will understand that the pressure-exerting means is an optional feature, and that gravity on its own will, in many cases, be sufficient to allow for the extraction of umbilical cord blood from the umbilical cord and/or placenta.

Once the blood source is correctly arranged, the closure 118, 218 is moved to its closed position and the latch or clip 120, 220, if provided, is secured to retain the closure on the housing 102, 202. The first collection means 116, 216 is connected to (e.g. screwed onto or pushed onto) the housing 102, 202 at the first output 114, 214 and the second collection means 138, 238 is arranged near the second output 136, 236. For example, the second collection means 138, 238 may be connected to (e.g. screwed onto or pushed onto) the housing 102, 202 at the second output 136, 236. The umbilical cord 106, 206 may be inserted into the second collection means 138, 238 so as to minimise spillage of umbilical cord blood.

Once the device has been assembled, the inflatable 124, 224 is inflated, for example using the pump 130, 230. As the inflatable 124, 224 expands, it exerts pressure on the blood source within the housing, thereby forcing blood out of the blood source. In the device 100 shown in FIG. 4, blood passes from the placenta 108 and through and/or along the umbilical cord 106, out of the second output 136, and into the second collection means 138 for storage. In the device 200 shown in FIG. 5, blood passes from the placenta 208 and through/along the umbilical cord 206, through the first output 214 into the first collection means 216 (but remaining within the umbilical cord), and then out of the second output 236 and into the second collection means 238 for storage.

In the embodiment shown in FIG. 4, the first output 114 is configured to allow passage of waste tissue and/or fluid from the placenta and/or umbilical cord therethrough. This waste then passes into the first collection means 116 and is disposed of.

In the embodiment shown in FIG. 5, the first output 214 is configured to allow passage of the umbilical cord and waste tissue and/or fluid from the placenta and/or umbilical cord therethrough. The umbilical cord then passes out of the first collection means 216 through the second output 236, leaving the waste in the first collection means 216 for disposal.

It is advantageous for the device 100, 200 of the present invention to be mountable on a piece of medical equipment e.g. hospital equipment or furniture. To facilitate mounting of the device, a retaining means 140, 240 in the form of a hook or hanger can optionally be provided on the device.

It is also advantageous for the device to be disposable, so that it can be supplied to medical institutions and/or practitioners as a single-use, easy to use kit e.g. single-use, non-reusable.

Kit for Active Infusion of Isolated Body Tissue

A kit is provided for carrying out the method of the present invention. The kit comprises a cryoprotectant; and an infusion means for actively infusing the tissue with the cryoprotectant.

When the body tissue is umbilical cord, the kit may optionally comprise a device for collecting or harvesting cord blood before the cord tissue is prepared for storage. This device may comprise a housing configured to receive a blood source and a first collection means in communication with the housing, wherein the housing comprises a first output configured to allow passage of blood extracted from the blood source and/or at least a portion of the blood source therethrough. Suitable umbilical cord blood collection devices are described above, in FIGS. 4 and 5, and in WO-A-2014/057353 (Virgin Health Bank QSTP-LLC, incorporated herein by reference).

The present invention has been described above in exemplary form with reference to the accompanying drawings which represent a single embodiment of the invention. It will be understood that many different embodiments of the invention exist, and that these embodiments all fall within the scope of the invention as defined by the following claims.

1. A method of preparing isloated body tissue for crygenic storage, the method comprising a step of actively infusing the body tissue with a cryoprotectant.

2. A method according to claim 1, wherein the body tissue comprises stem cells.

3. A method according to claim 1 or claim 2, wherein the body tissue is actively infused with the cryoprotectant using a infusion means.

4. A method according to claim 3, wherein the infusion means comprises a pressure-exerting means.

5-20. (canceled)

21. A method of storing umbilical cord tissue, comprising:
actively infusing umbilical cord tissue with a cryoprotectant;
cryogenically storing the cryoprotectant-infused umbilical cord tissue for at least one year.

22. A method according to claim 21, further comprising the steps of:
thawing all or part of the stored umbilical cord tissue; and
isolating stem cells form the thawed umbilical cord tissue.

23. A kit for preparing umbilical cord tissue for cryogenic storage, comprising:
a cryoprotectant; and
infusiong means for actively infusing the umbilical cord tissue with the cryoprotectant.

24. A kit according to claim 23, wherein the infusion means comprises a pressure-exerting means.

25. A kit according to claim 23 or 24, comprising (iii) a device for collecitng or harvesting blood comprising a housing configured to receive a blood source and a first collection means in communication with the housing, wherein the housing comprises a first output configured to allow passage of blood extracted from the blood source and/or at least a portion for the blood source therethrough.

26. A kit according to claim 25, wherein the device is configured to allow for collection of blood from the blood source using a gravitational force.

27-28. (canceled)
